(19) United States
(12) Patent Application Publication
(10) Pub. No.: US 2019/0040344 A1
(43) Pub. Date: Feb. 7, 2019

(54) DEVICE AND METHOD FOR SAMPLE ANALYSIS

(71) Applicant: Scanogen Inc., Baltimore, MD (US)

(72) Inventors: Alfredo A. Celedon, Columbia, MD (US); John W. Pettit, Derwood, MD (US)

(21) Appl. No.: 16/047,398

(22) Filed: Jul. 27, 2018

Related U.S. Application Data
(60) Provisional application No. 62/537,602, filed on Jul. 27, 2017.

(51) Int. Cl. C12M 1/00; C12Q 1/04; C12M 3/06; B01L 3/00

(52) U.S. Cl. CPC C12M 1/007; C12Q 1/04; C12M 23/16; B01L 3/5027; C12M 23/58; C12M 23/38; C12M 23/14

(57) ABSTRACT

The present invention describes a device used to process a sample and detect the presence or absence of certain molecules in the sample. The device includes a pouch having two or more compartments that may be created by sealing two or more films. In a preferred embodiment, the pouch is made by a single sheet of film that has been folded onto itself. In another preferred embodiment, the pouch is made by sealing two films. In other aspects, this invention describes methods to process a sample.

### CROSS REFERENCE TO RELATED APPLICATIONS

The present application claims the benefit of U.S. Provisional Application No. 62/537,602 filed Jul. 27, 2017, the contents of which are incorporated by reference herein in their entirety.

### STATEMENT REGARDING FEDERALLY SPONSORED RESEARCH OR DEVELOPMENT

This invention was made with government support under Grants No. R44A1122527 and R43A1124871 awarded by the National Institutes of Health. The government has certain rights in the invention.

### FIELD OF THE INVENTION

The present invention relates to devices and methods for analyzing samples and detecting the presence of certain molecules in the sample.

### BACKGROUND OF THE INVENTION

There is a need for easy to operate and self-contained systems for the detection of molecules in a sample, for example detecting biomolecules in body fluid samples, which can be used in diagnosis of infectious diseases and early detection of cancer. Another example is detection of biomolecules in environmental samples to detect the presence of potentially dangerous microorganisms, such as anthrax.

However, most sensitive detection systems are difficult to operate and not self-contained. For example, PCR-based detection systems often require the user to conduct manually one or more sample preparation steps that involve pipetting before beginning automated steps. In addition, most sensitive detection systems are not self-contained meaning that not all reagents are integrated in a single device.

In addition, detection systems should minimize the contact of the sample with the environment. However, samples and reagents come into contact with the environment (for example, environmental air) in most detection systems thereby increasing the possibility of false-positive results due to contamination. In addition, the fact that the sample is exposed to the environment usually makes the system more difficult to operate. For example, to decrease the possibility of contamination some systems require the user to disinfect the work area before operation. Systems that do not minimize sample contact with the environment also increase the possibility of releasing potentially infective agents into the environment.

The present invention provides a solution to the problems mentioned above by providing devices and methods that can be automated and therefore are easy to operate. I addition, the devices and methods minimize the exposure of samples and reagents with the environment.

### SUMMARY OF THE INVENTION

In one aspect, the present invention is directed to a pouch for use in a process for detecting the presence or concentration of a target analyte in a sample, wherein the pouch comprises:

a) one or more compartments capable of holding a fluid; and

b) one or more flow channels having at least two surfaces, wherein one flow channel i) connects two compartments within the pouch to permit fluid to flow from one compartment to another, and/or ii) connects one compartment of the pouch with the exterior of the pouch to allow fluid to flow from the compartment to the exterior of the pouch;

wherein at least one of the flow channels has a shape such that the channel can be closed with a single device that exerts pressure on such channel.

In another aspect, the present invention is directed to a pouch for use in a process for detecting the presence or concentration of a target analyte in a sample, wherein the pouch comprises:

a) a first compartment in first fluid connection with a first fluidic connector, wherein the first compartment is capable of holding a fluid and wherein fluid is capable of flowing between the first fluidic connector and first compartment; and

b) a second compartment in second fluid connection with a second fluidic connector, wherein the second compartment is capable of holding a fluid and wherein fluid is capable of flowing between the second fluidic connector and second compartment;

wherein the first and second fluidic connectors are positioned so as to be capable of simultaneously communicating with a fluid test sample.

In yet another aspect, the present invention is directed to a pouch for use in a process for detecting the presence or concentration of a target analyte in a sample, wherein the pouch comprises:

a) a first compartment in first fluid connection with a container external to the pouch, wherein the first compartment is capable of holding a fluid and wherein fluid is capable of flowing between the container and the first compartment; and

b) a second compartment in second fluid connection with the container, wherein the second compartment is capable of holding a fluid and wherein fluid is capable of flowing between the container and the second compartment;

wherein the first and second fluid connections are positioned so as to be capable of simultaneously communicating with a fluid that may be present in the container.

In yet another aspect, the present invention is directed to a pouch formed from one or more pieces of film, capable for use in a process for detecting the presence or concentration of a target analyte in a sample, wherein the pouch comprises:

a) a first region where two portions of film have been sealed together, the first region having a first and second side; and

b) a second region defining a channel where the two portions of film have not been sealed together, the second region having a first and second side; and

c) a third region that defines a portion of the channel, wherein two sides of the channel are formed from one or more pieces of a film having a greater rigidity than the portions of film that form the first and second regions.

In yet another aspect, the present invention is directed to a method for loading a sample from a container into a pouch, the method comprising:

a) providing a pouch having a first compartment in first fluid connection with a first fluidic connector and a second compartment in second fluid connection with a second fluidic connector wherein the first compartment is capable of holding a fluid and wherein fluid is capable of flowing between the first fluidic connector and the first compartment, and wherein the second compartment is capable of holding a fluid and wherein fluid is capable of flowing between the second fluidic connector and the second compartment;

b) connecting the first and second fluidic connectors from the pouch to fluidic connectors fluidically connected to the interior of an external container that contains a sample; and

c) applying pressure to the first compartment to initiate flow of material from the first compartment into the external container and to initiate flow of material from the external container into the second compartment.

In yet another aspect, the present invention is directed to a method for loading a sample from a container into a pouch, the method comprising:

a) providing a pouch comprising a first compartment in first fluid connection with a first hollow needle, wherein the first compartment is capable of holding a fluid and wherein fluid is capable of flowing between the first hollow needle and first compartment, and a second compartment in second fluid connection with a second hollow needle, wherein the second compartment is capable of holding a fluid and wherein fluid is capable of flowing between the second hollow needle and second compartment;

b) providing a container than contains a sample;

c) penetrating the container with the first and second needles; and

d) applying pressure to the first compartment to initiate flow of material from the first compartment into the external container and to initiate flow of material from the external container into the second compartment.

In yet another aspect, the present invention is directed to a method for loading a sample from a container into a pouch, the method comprising:

a) providing a pouch comprising a first compartment in first fluid connection with a container external to the pouch having a sample, wherein the first compartment is capable of holding a fluid and wherein fluid is capable of flowing between the container and the first compartment, and a second compartment in second fluid connection with the container, wherein the second compartment is capable of holding a fluid and wherein fluid is capable of flowing between the container and the second compartment; and
b) applying pressure to the first compartment to initiate flow of material from the first compartment into the external container and to initiate flow of material from the external container into the second compartment.

In yet another aspect, the present invention is directed to a method to reduce the volume and exchange the liquid in which a magnetic particle is suspended, the method comprising:

a) providing a compartment having a flow channel, wherein the compartment contains an initial volume of liquid with at least one magnetic particle;
b) placing a magnet in sufficient proximity to the compartment to attract the magnetic particle, optionally waiting until the particle has reached a region close to the magnet;
c) reducing the volume of the compartment to remove at least a part of the liquid from the compartment while keeping the magnetic particle within the compartment;
d) constraining at least part of the compartment such that the maximum volume that the compartment can reach is less than the initial volume of liquid;
e) adding fresh liquid to the constrained compartment; and
f) causing the liquid and the magnetic particle to leave the compartment.

These and other aspects of the present invention are described below with reference to the drawings and the detailed description.

### BRIEF DESCRIPTION OF THE FIGURES

FIG. 1A shows a pouch (1) with two compartments connected by an inverted “U” shaped flow channel (3).

FIG. 1B shows a pouch (101) with a clamp (105) that closes the inverted “U” shaped flow channels (103) and (104) that connect compartments in a three-compartment pouch. The “U” shaped channels (103) and (104) can be closed with a single clamp (105).

FIG. 2A shows a pouch (201) with multiple compartments made by heat sealing two plastic films. The heat-sealed areas are shown with diagonal hatching or with short vertical lines (212) and (213), while areas that are not heat sealed are shown white. The diagonal-hatching areas correspond to the initial heat seal while the short vertical lines (212) and (213) represent regions that are sealed after loading some compartments with reagents. The pouch has a releasable clamp bar (206) that closes some flow channels to prevent transfer of reagents between compartments during storage and transportation. The pouch has holes (205) that are used for pouch alignment in the instrument. The pouch has also a fitment (207) for connection to an external container. The external container may contain the sample that is loaded into the pouch. The fitment may be heat sealed to the pouch so that two tubes of the fitment are connected to flow channels from compartment (202) and (203).

FIG. 2B shows fitment (207). The fitment has two hollow needles (208) that are used to perforate the external container. The needles may be connected to tubes (209) for improved sealing to the pouch.

FIG. 3 shows a fitment (207) with two needles (208) that are connected to compartments (202) and (203) of a pouch. Some flow channels in the pouch have been closed by applying external pressure to the channels (301). Container (302) has a rubber membrane (303) that seals the container and can be penetrated by the needles (208) so that both compartments (202) and (203) become fluidically connected to the interior of the external container (302).

FIG. 4 shows an external container (405) connected to a pouch. The external container contains the sample and has a cap that is air tight. The pouch has been formed by heat sealing two films (diagonal-hatching regions and regions with horizontal lines (412)). Two tubes (409) fluidically connect the interior of the container to compartments (402) and (403) via flow channels in the pouch. The tubes (409) are heat sealed to the pouch. The external container (405) is well sealed from the exterior, so that if pressure is increased in compartment (402) and material flows into the external container (405), then the pressure in the external container (405) increases and material from the interior of external container (405) may flow into compartment (403).

FIGS. 5A-5E show an example of this invention for concentrating magnetic particles and the molecules attached to magnetic particles. FIGS. 5A-5E depict a step by step process. Not all the steps are always necessary, and the order of the steps may be different to the one shown here. This invention includes different combination of the steps listed below.

FIG. 5A shows a pouch compartment (512) made of a first film (510) and a second film (511). The compartment contains a liquid with magnetic particles (513).

FIG. 5B shows a magnet (514) that is placed close to the compartment (512) and attracts the magnetic particles (513).

FIG. 5C shows a displacement of the magnet (514) which displaces the particles (513).

FIG. 5D shows an external depressor (515) which reduces the volume of the compartment (512).

FIG. 5E shows a displacement of the magnet (514) which further reduces the volume of the compartment (512).

FIG. 6 shows an example of an embodiment of a flow channel of this invention created in a pouch region where two films (605) and (608) are sealed together and where the flow channels have at least one rigid surface. A piece of rigid material (606) between two films (605) and (608) forms a flow channel (607). The black discs (609) indicate regions of bonding between the films (605) and (608) and the piece of material (606).

FIG. 7A shows an example of an embodiment of a flow channel of this invention created in a pouch region (701) with two film surfaces. The pouch region (701) is made by heat sealing two films (diagonal-hatching regions). A channel (702) is formed in the area that is not heat sealed. In order to replace the film surfaces of the capillary with two rigid pieces, the following is done: a rectangular window is cut in the pouch (704) (the two films are cut) and two rectangular pieces are sealed to the outside of the pouch (the contour of the rigid pieces is (703)), one piece at each side of the pouch. The pieces are (708) and (709) in section AA view in FIG. 7B. The area between (703) and (704) is where the pieces are glued to the film. Preferably, double sided tape (tape with glue at both sides) is used to give the desired deepness to the capillary (710).

FIG. 7B shows view of section AA of the capillary. The pouch region is made with two films (705) and (706). The film pieces that are cut out from the original pouch are replaced with two rigid pieces (708) and (709) that are glued to films (705) and (706) using a material (707), such as double-sided tape that may help increase and select a desired deepness for the capillary (710).

FIG. 8 is a view of a multi-compartment flexible pouch with 10 compartments (801) to (810). The pouch is made by sealing first the regions shown in back, then loading some compartments with reagents, that may be gas, liquid or solid, removing excess gas, and finally sealing the regions that are shown with short vertical lines (812) and (813). A clamp may be used to close flow channels during transportation and storage. Compartments (801) and (802) are both connected to the interior of an external container (820). The external container (820) is well sealed from the exterior, so that if pressure is increased in compartment (801) and material go into the external container (820), then the pressure in the external container (820) increases and material from the interior of external container (820) may go into compartment (802).

FIGS. 9A-9D show an example of this invention for concentrating magnetic particles and the molecules attached to magnetic particles. FIGS. 9A-9D depict a step by step process. Not all the steps are always necessary, and the order of the steps may be different to the one shown here. This invention includes different combination of the steps listed below.

FIG. 9A shows a pouch compartment (901) over a solid support (903). The compartment contains a liquid with magnetic particles (902).

FIG. 9B shows a magnet (905) that is placed close to the compartment and attracts the magnetic particles. Depressors (904) apply pressure to the compartment to remove all or part of the liquid in the compartment.

FIG. 9C shows that the compartment has been substantially constrained between the solid support and the depressors.

FIG. 9D shows that some of the spatial constrains on the compartment have been removed, in this case by removing one of the depressors while one depressor remains in place. The maximum volume of liquid that the compartment can hold is significantly reduced with this constrain. Liquid flows into the compartment and the magnet can be removed to resuspend the particles.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The terms “about” and “substantially” are used herein to mean approximately, in the region of, roughly, or around. When the terms “about” and “substantially” are used in conjunction with a numerical range, it modifies that range by extending the boundaries above and below the numerical values set forth. In general, the terms “about” and “substantially” are used herein to modify a numerical value above and below the stated value by a variance of less than about 20%.

The terms “target analyte” or “analyte,” are used herein to denote the molecule to be detected in the test sample. According to the invention, there can be any number of different target analytes in the test sample (from one to one thousand, or even more). The target analyte can be any molecule for which there exists a naturally or artificially prepared specific binding member. Examples of target analytes include, but are not limited to, a nucleic acid, oligonucleotide, DNA, RNA, protein, peptide, polypeptide, amino acid, antibody, carbohydrate, lipid, hormone, steroid, toxin, vitamin, any drug administered for therapeutic and illicit purposes, a bacterium, a virus, cell, as well as any antigenic substances, haptens, antibodies, metabolites, water pollutants (such as nitrates, phosphates, heavy metals, etc.) and molecules having an odor, such as compounds containing sulfur and/or nitrogen, for example hydrogen sulfide, ammonia, amines, etc., and combinations thereof.

The terms “test sample” or “sample” are used interchangeably herein and include, but are not limited to, biological and environmental samples that can be tested by the methods of the present invention described herein and include human and animal body fluids such as whole blood, serum, plasma, cerebrospinal fluid, urine, lymph fluids, and various external secretions of the respiratory system (such as sputum), intestinal and genitourinary tracts, tears, saliva, milk, white blood cells, myelomas and the like, biological fluids such as cell culture supernatants, fixed tissue specimens and fixed cell specimens, PCR amplification products or a purified product of one of the above samples. Suspensions of cells or biomolecules that can be produced from a swab from a human, animal or an environmental surface. A “sample” may include gaseous mediums, such as ambient air, chemical or industrial intermediates, chemical or industrial products, chemical or industrial byproducts, chemical or industrial waste, exhaled vapor, internal combustion engine exhaust, or headspace vapor such as vapor surrounding foods, beverages, cosmetics, vapor surrounding plant or animal tissue and vapor surrounding a microbial sample. Another example of “sample” relevant to this invention is a liquid solution produced by dissolving material collected by filtering a gaseous sample or a liquid solution produced by exposing the liquid to a gaseous sample. Additional sample mediums include supercritical fluids such as supercritical CO₂ extricate. Other exemplary mediums include liquids such as water or aqueous solutions, oil or petroleum products, oil-water emulsions, liquid chemical or industrial intermediates, liquid chemical or industrial products, liquid chemical or industrial byproducts, and liquid chemical or industrial waste. Additional exemplary sample mediums include semisolid mediums such as animal or plant tissues, microbial samples, or samples containing gelatin, agar or polyacrylamide.

The present invention relates to a pouch used to process a sample for detection of one or more target analytes. The pouch has two or more compartments created by sealing two or more films. In a preferred embodiment, the pouch is made by a single sheet of film that has been folded onto itself. In another preferred embodiment, the pouch is made by sealing two films.

The term compartment is used to identify a region of the pouch that is fluidly connected with other regions inside or outside the pouch. In preferred embodiments, the compartment is sealed around mostly using a permanent seal with one or more flow channels connected to it. In preferred embodiments, a compartment is used to store materials or reagents.

The term material or reagents is used to indicate gases, liquids and solids materials that participate in the processes performed using the pouch.

The term fluid is used to indicate a gas or a liquid.

The term channel or flow channel is used to identify a region of the pouch that is connected to one or more compartments, allowing passage of materials and reagents in and/or out of the compartment. In preferred embodiments, a flow channel can be sealed by a reversible method, such as a clamp, plug, stopper or frangible (burstable) seal.

The term clamp is used to indicate a device capable of applying external pressure on one or more flow channels substantially reducing or eliminating the capacity of the one or more flow channels to transfer materials.

A type of pouch of this invention has two or more compartments comprising a first flexible film disposed against a second layer that can also be a flexible film, the same layer of film folded against itself, or a thicker or stiffer film. The two layers are bonded to each other by heat seals or other means known in the art including ultrasonic welding or gluing. The shape of the bond creates compartments that can be used to hold materials, as well as create flow channels that interconnect the individual compartments or bring material into or out of the pouch.

A self-contained detection system where all the reagents required for detection are included in a single device should prevent the different reagents from mixing with each other during transportation and storage. This function is particularly critical if some of the reagents are gases or liquids. Burstable seals have been used to force reagents to remain within a compartment during transportation and storage. These seals are open during operation by applying a predetermined pressure on the fluid path (see U.S. Pat. No. 5,674,653). However, burstable seals can be difficult to produce in a reliable manner. Releasable clamps that apply pressure to connecting channels can be used to prevent passage of materials through a channel during transportation and storage. However, the more channels that need to be closed, the more impractical this approach is. In one embodiment, the present invention provides a flow path between compartments that resolves this problem. According to this embodiment, two or more channels in a pouch have an inverted “U” shape, proceeding upwards and then returning downward to connect to another compartment or to connect with the outside of the pouch.

Two or more “U” shaped channels can be sealed with a single releasable clamp significantly reducing the complexity of closing multiple channels during transportation and storage.

The term “U” shaped flow channel or “U” shaped channel is used to indicate a channel that rises above one or more compartments that it connects to. It should be clear that despite the use of the term “U” shaped to describe these channels, the actual shape of the channels may be any arbitrary shape that meets the requirement of rising above one or more compartments that are connected to. The “U” shaped channel may connect a single compartment with the outside of the pouch. In this case, the channel is considered a “U” shaped channel if it rises above the level of the single compartment.

The pouches of this invention may have at least two flow channels disposed such that they can be sealed shut by a single means, such as a clamp. When the clamp is removed, the flow channels will open from fluid pressure allowing fluid to flow through them when it is desired to move fluid from one compartment to another. Preferably, the clamp can be removed during use to allow movement of the materials in the compartments and then replaced after use to seal the compartments for safe disposal.

The pouches of this invention may be made by a process that includes a first step of heat sealing or other sealing methods known in the art, wherein the first step of sealing leaves portions of each compartment along one or more sides of the pouch that are not sealed so that the compartments may be individually filled with their own respective fluid or material and be purged or emptied of any air or gas remaining in the compartment if desired. After filling each compartment, excess gas may be removed from compartments. When the filling and purging operation is complete, the sides that were used for filling can be sealed.

In another embodiment, this invention provides devices and methods to minimize the exposure of sample and reagents to the environment when a sample is transferred from a container into the pouch. According to this embodiment, a pouch of this invention may have a first compartment that contains a liquid or a gas. The first compartment has a first flow channel that can be fluidly connected to a reservoir that contains the sample. The connection may be through a first hollow needle with a sharp tip that can penetrate a wall in the reservoir that contains the sample. More generally, the connection may be by any permanent or releasable fluidic connector. The reservoir that contains the sample is air tight during the transfer process. The reservoir that contains the sample may have a cap that is closed after the sample is added to the reservoir. A second compartment in the pouch has a second flow channel that is also fluidly connected to the reservoir that contains the sample. The connection may be through a second needle disposed adjacent to the first needle such that they can simultaneously puncture the seal of the container. More generally, the connection may be by any permanent or releasable fluidic connectors. If the first and second compartments are connected to the sample reservoirs through first and second needles, then preferably, the first and second needles are held in place by a rigid structure, preferably, the needles are substantially parallel to each other, and preferably, the needles are between 1 and 30 millimeters apart from each other.

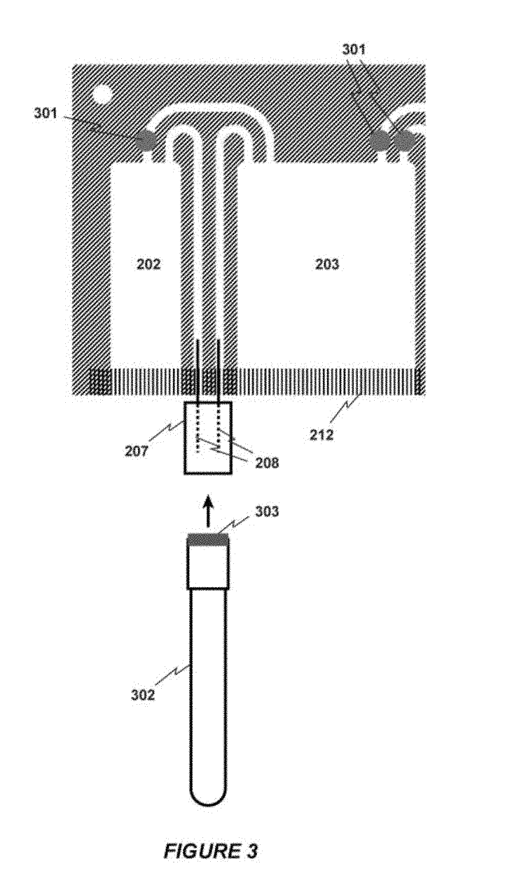

In one aspect of this embodiment, the first compartment contains a gas. The gas in the first compartment can be expelled into the sample reservoir by increasing the pressure in the compartment. The gas displaces the contents of the sample container forcing them out through the second channel and into the second compartment. A predetermined volume of material can be thereby brought into the second compartment with minimal exposure of the sample to the environment. FIG. 3 shows a pouch with compartments (202) and (203) which can be connected to an external container (302) by using the needles (208) which are fluidically sealed to channels connected to (202) and (203). The needles can penetrate a rubber membrane (303) in the external container (302). When the needles (208) penetrate the external container both compartments (202) and (203) become fluidically connected to the interior of the external container (302). Containers with a rubber membrane useful for this invention are known in the art. A common example is the Vacutainer®. A Vacutainer® blood collection tube is a sterile glass or plastic test tube with a colored rubber stopper creating a vacuum seal inside of the tube, facilitating the drawing of a predetermined volume of liquid. Vacutainer® tubes may contain additives designed to stabilize and preserve the specimen prior to analytical testing. Tubes are available with a safety-engineered stopper, with a variety of labeling options and draw volumes. The color of the top indicates the additives in the vial. Vacutainer® is a registered trademark of Becton Dickinson, which manufactures and sells the tubes today. By using devices and methods of this invention, the contents of a Vacutainer®, or other containers may be brought to the interior of a pouch with minimal exposure of the sample to the environment.

The term fluidic connection, fluidic connector and fluidic connectors are used to indicate a connection between two parts of a fluidic path, or each of the components that can be used to make a connection between two parts of a fluidic path. Many fluidic connectors, both permanent and releasable relevant to the present invention are known in the art. An example of a permanent connection is the connection of a plastic tubing that is heat-sealed to the flow channel of a pouch. A releasable connection usually comprises two fluidic connectors, one located at each side of the fluidic path being connected. One example of releasable connection is a connection made between a hollow needle on one side of the fluidic path and a rubber membrane on the other side of the fluidic path. When the needle penetrates the rubber membrane, a fluidic connection can be established. Another example of releasable connectors are Luer taper connections. The Luer taper is a standardized system of small-scale fluid fittings used for making leak-free connections between a male-taper fitting and its mating female part on medical and laboratory instruments, including hypodermic syringe tips and needles or stopcocks and needles. Currently ISO 80369 governs the Luer standards and testing methods. There are two varieties of Luer taper connections: locking and slipping. An example of sample reservoir relevant for this embodiment may have two Luer taper connectors fluidically connected to the interior of the sample reservoir, for example, two female slipping Luer connectors. This pair of connectors can be connected to a mating pair of slipping Luer male connectors fluidically connected to the first and second compartment in the pouch.

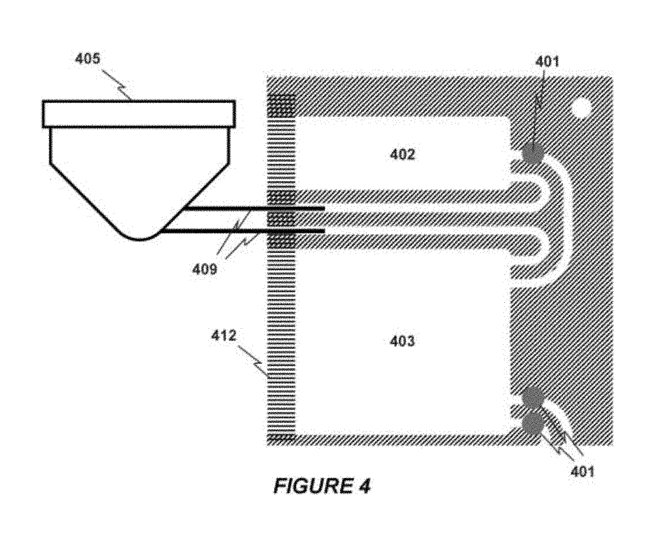

In another aspect of this embodiment, the first compartment contains a liquid that can be for example a sample preparation reagent that needs to be mixed with the sample. The liquid in the first compartment can be expelled into the sample reservoir by increasing the pressure in the compartment. The liquid from the first compartment mixes with the sample in the sample container and the increased pressure in the sample container forces some of the contents of the container out through the second channel and into the second compartment. Optionally, the pressure in the second compartment can then be increased so that contents of the second compartment return to the sample reservoir. The increased pressure in the sample container can force some of the contents of the container out through the first channel and into the first compartment. By alternating the application of pressure to the first and second compartment, the sample can be mixed with the liquid originally present in the first compartment and a predetermined volume of material can be also brought into the detection device with minimal exposure of the sample and reagents to the environment. FIG. 4 shows a pouch with compartments (402) and (403) which are fluidically connected to an external container (405) that may contain a sample. Two tubes (409) are sealed to channels from (402) and (403) and fluidically connect to the interior of the external container (409).

In order to fill a compartment of a pouch of this invention with a precise volume of gas, a solid structure can be inserted into the first compartment. The solid structure preferably satisfies two criteria: First, it should allow gas to remain in the compartment once the compartment is sealed. Second, the structure deforms or breaks under sufficient force thereby allowing gas to be expelled from the compartment when needed. Examples of solid structures suitable for this invention are: sponges, deformable and hollow structures made of plastic, and bended sheets of paper.

As used herein, the terms film, plastic film, or sheet are used to indicate a material that has been shaped into a substantially flat object. The thickness of a film of this invention ranges from about 0.3 to about 5,000 microns. Some films of the present invention are formed by a manufacturing processes such as cast film, extruded film or blown film. A film may comprise one of more layers of plastic, metals and papers. Examples of plastics suitable for this invention include polyethylene, polypropylene, cast polypropylene, polyvinyl chloride (PVC), nylon, polyester, polystyrene, Teflon® and cellophane. A metal layer is usually composed of aluminum. A thin layer of a metal such as aluminum can be deposited on a film to generate a metallized film. Films with multiple layers can be produced by co-extrusion. Suryln®, produced by Dupont, is an ionomer resin available for use in conventionally blown and cast film extrusion and co-extrusion equipment. Some films of the present invention may be rigid. Some films of the present invention may have a thickness ranging between 200 and 5,000 microns.

Heat sealing is the process of sealing one thermoplastic to another similar thermoplastic using heat and pressure. The direct contact method of heat sealing utilizes a constantly heated die or sealing bar to apply heat to a specific contact area or path to seal or weld the thermoplastics together. Heat sealing is used for many applications, including heat seal connectors, thermally activated adhesives, film media, plastic ports or foil sealing.

Heat sealing has several common applications. Heat sealing of products with thermal adhesives is used to hold clear display screens onto consumer electronic products and for other sealed thermo-plastic assemblies or devices where heat staking or ultrasonic welding are not an option due to part design requirements or other assembly considerations.

Heat sealing also is used in the manufacturing of blood test film and filter media for the blood, virus and many other test strip devices used in the medical field today. Laminate foils and films often are heat sealed over the top of thermoplastic medical trays, Microtiter (microwell) plates, bottles and containers to seal and/or prevent contamination for medical test devices, sample collection trays and containers used for food products.

Heat sealing is used to produce medical and fluid bags used in the medical, bioengineering and food industries. Fluid bags are made out of a multitude of varying materials such as foils, filter media, thermoplastics and laminates.

Hot bar sealers have heated tooling kept at a constant temperature and is also known as Direct Contact Thermal Sealing. They use one or more heated bars, irons, or dies which contact the material to heat the interface and form a bond. The bars, irons and dies have various configurations and can be covered with a release layer or utilize various slick interposer materials such as Teflon films to prevent sticking to the hot tooling.

Continuous heat sealers, also known as Band type heat sealers, utilize moving belts over heating elements.

Impulse heat sealers are not continuously heated; heat is generated only when electric current flows through a heating element in the sealers. When the materials are placed in the heat sealer, they are held in place by pressure. An electric current heats the heating element for a specified time to create the required temperature. The jaws hold the material in place after the heat is stopped, sometimes with cooling water; this allows the material to fuse before stress can be applied. Hot melt adhesive can be applied in strips or beads at the point of joining. It can also be applied to one of the surfaces during an earlier manufacturing step and reactivated for bonding. Hot wire sealing involves a heated wire that both cuts the surfaces and joins them with a molten edge bead. This is not usually employed when barrier properties are critical. Induction sealing is a non-contact type of sealing used for inner seals in bottle caps.

Induction welding heat seals by inducing a current in a conducting layer through the use of non-contact electro-magnetic induction heating. Ultrasonic welding uses high-frequency ultrasonic acoustic vibrations applied to workpieces being held together under pressure to create a weld.

A type of heat sealer is also used to piece together plastic side panels for light-weight agricultural buildings such as greenhouses and sheds. This version is guided along the floor by four wheels.

Good seals are achieved as a result of time, temperature and pressure being correctly set for the type of clean material being employed.

The type of film used to form the pouch of this invention may be one of numerous varieties of film used in medical, pharmaceutical and food packaging processes. One suitable choice is film made from polyethylene terephthalate (PET) that has been stretched in both the machine as well as cross-machine directions, or Biaxially Stretched (BOPET) film. This film is offered by numerous manufactures, such as DuPont who markets this film under the trade names Mylar® or Melinex®. Other examples of suitable film Biaxially Stretched Polypropylene (BOPP), metallized BOPP, low density polyethylene (LDPE), Suryln®, and films generated by laminating combinations thereof.

In order to make BOPET film be heat-sealable, a rather thin layer of amorphous PET (APET or aPET) is added to the PET in a co-extrusion process. BOPET films with an APET heat seal layer are used in many packaging applications. This film can be made to seal to itself, has rather good barrier properties, can be obtained optically clear and is capable of withstanding quite high temperature in the range of 250 degrees Centigrade.

Several standard test methods are available to measure the strength of seals. In addition, package testing is used to determine the ability of completed packages to withstand specified pressure or vacuum. Several methods are available to determine the ability of a sealed package to retain its integrity, barrier characteristics, and sterility.

Sealing processes can be controlled by a variety of quality management systems such as HACCP, statistical process control, ISO 9000, etc. Verification and validation protocols are used to ensure that specifications are met and final materials/packages are suited for end-use. The efficacy of seals is often detailed in governing specifications, contracts, and regulations. Quality management systems sometimes ask for periodic subjective evaluations: For example, some seals can be evaluated by a simple pull to determine the existence of a bond and the mechanism of failure. With some plastic films, observation can be enhanced by using polarized light, which highlights the birefringence of the heat seal. Some seals for sensitive products require thorough verification and validation protocols that use quantitative testing.

There are several test methods used to establish the strength of a seal. Seal Strength testing, also known as Peel Testing, measures the strength of seals within flexible barrier materials. This measurement can then be used to determine consistency within the seal, as well as evaluation of the opening force of the package system. Seal strength is a quantitative measure for use in process validation, process control and capability. Seal strength is not only relevant to opening force and package integrity, but to measuring the packaging processes' ability to produce consistent seals.

The burst test is used to determine the package's strength and precession. The burst test is performed by pressurizing the package until it bursts. The results for the burst test include the burst pressure data and a description of where the seal failure occurred. This test method covers the burst test as defined in ASTM F1140. The creep test determines the package's ability to hold pressure for an extended period. The creep test is performed by setting the pressure at about 80% of the minimum burst pressure of a previous burst test. The time to seal failure or a pre-set time is measured to determine if the test is passed or failed.

Some films of this invention may be thermoformed. Thermoforming is a manufacturing process where a plastic sheet is heated to a pliable forming temperature, formed to a specific shape in a mold, and trimmed to create a usable product. The sheet or film is heated in an oven to a high-enough temperature that permits it to be stretched into or onto a mold and cooled to a finished shape. Its simplified version is vacuum forming. Vacuum forming is a simplified version of thermoforming, where a sheet of plastic is heated to a forming temperature, stretched onto a single-surface mold, and forced against the mold by a vacuum. This process can be used to form plastic into permanent objects such as turnpike signs and protective covers. Normally draft angles are present in the design of the mold (a recommended minimum of 3 degrees) to ease removal of the formed plastic part from the mold.

Films relevant to this invention may be produced by injection molding. Injection molding is a manufacturing process for producing parts by injecting material into a mold. Injection molding can be performed with a host of materials mainly including metals, (for which the process is called die-casting), glasses, elastomers, confections, and most commonly thermoplastic and thermosetting polymers. Material for the part is fed into a heated barrel, mixed, and forced into a mold cavity, where it cools and hardens to the configuration of the cavity. After a product is designed, usually by an industrial designer or an engineer, molds are made by a mold-maker (or toolmaker) from metal, usually either steel or aluminum, and precision-machined to form the features of the desired part. Injection molding is widely used for manufacturing a variety of parts, from the smallest components to entire body panels of cars.

Films and materials of this invention may be produced by cold forming. Cold forming is the process of forging metals at near room temperatures. In cold forming, metal is formed at high speed and high pressure into tool steel or carbide dies. The coldworking of the metal increases the hardness, yield, and tensile strength.

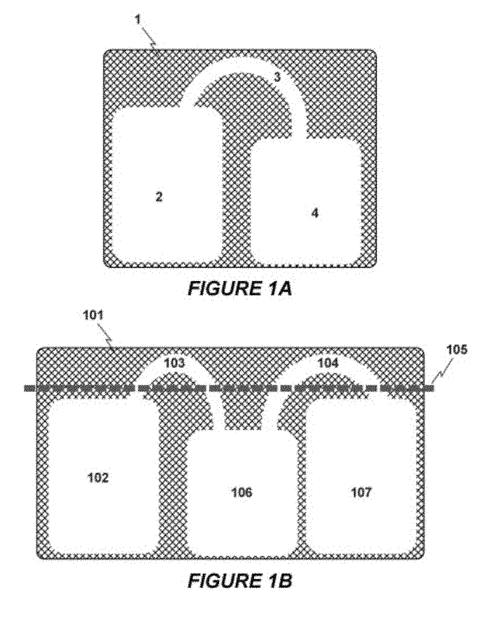

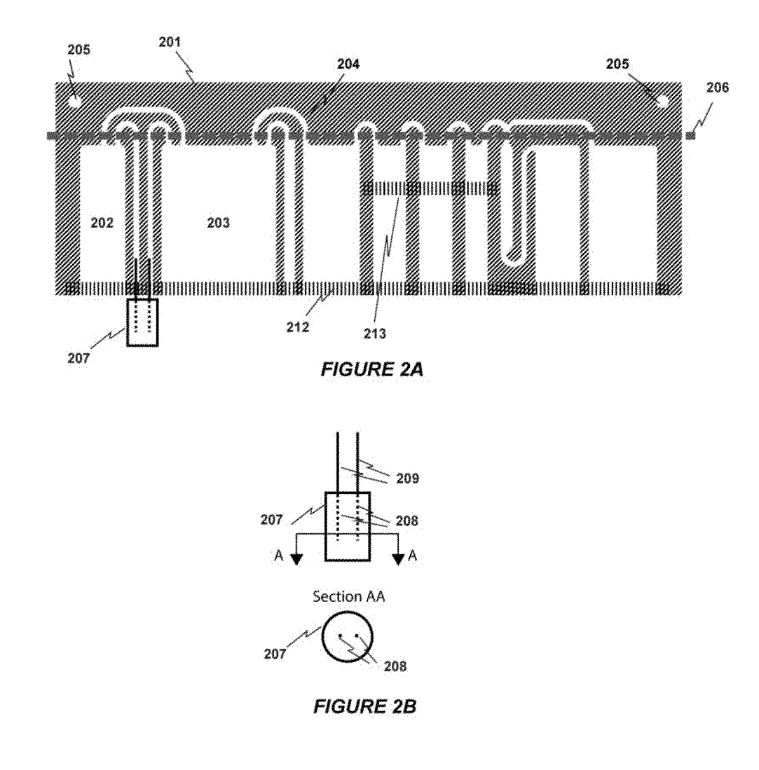

FIG. 1 shows two layers of plastic film sealed together to make a pouch that is often flexible. Item 1 in FIG. 1 is the region of the pouch that has been permanently sealed together (diagonal-hatching regions). Item 2 is a compartment that is formed in a region where the two films are not sealed together. FIG. 1B shows a pouch with three compartments connected by two “U” shaped channels (103) and (104) disposed in such a way that both channels can by closed with a single clamp (105). The clamp (105) closes the flow channel (103) and (104) by pressing the two film layers together during storage and shipment to prevent the escape of materials from the compartment into which the material was placed during manufacture.

This invention includes a clamp that seals the compartments during storage and shipment. At least two flow channels are aligned so that a single clamp crosses each one of them and can close each flow channel when the clamp is in place.

The design allows filling the compartments from the top or bottom and then a final heat sealing step completes the fabrication process. Air is often pulled out of a compartment before final sealing to prevent the occurrence of bubbles.

Flow channels may be designed to be comparatively long and thin with a tendency to stay closed, owing to the stiffness of the plastic film. Additional pressure from the clamp assures that they will remain closed during storage and shipment and prevent material leakage from one compartment to another or from one compartment to the exterior of the pouch.

When the pouch is in use, the clamp is removed and fluid pressure opens a selected flow channel to permit fluid to flow. Flow is generated by applying pressure to a compartment or to a flow channel. Flow can also be stopped by applying pressure to a flow channel. Various means of applying pressure to a compartment or to a flow channel would be apparent to one of ordinary skill (e.g., U.S. Pat. Nos. 4,673,657, 3,036,894, 9,102,911, and 8,735,055, incorporated herein by reference). Individual flow channel actuators, not shown, may be used to apply pressure to points along the respective flow channel to keep that flow channel from allowing fluid to flow.

The present invention may include a system and method including a compartment in a pouch and depressors. The compartment is depressed by means of a system of two or more depressors, where each individual depressor only acts on a portion of the surface area of the compartment. This system of two or more depressors may be used to depress all, some or none of the surface area of the compartment. By means of sequentially operating the depressors, the volume of the compartment may be adjusted or controlled.

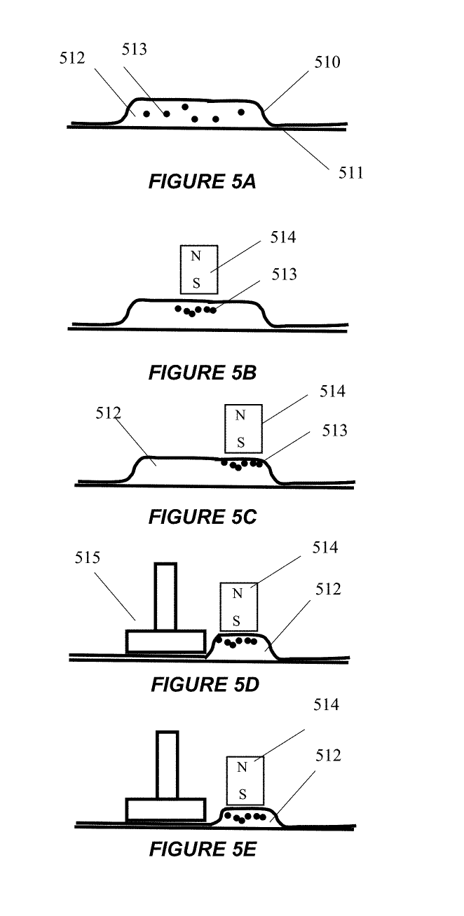

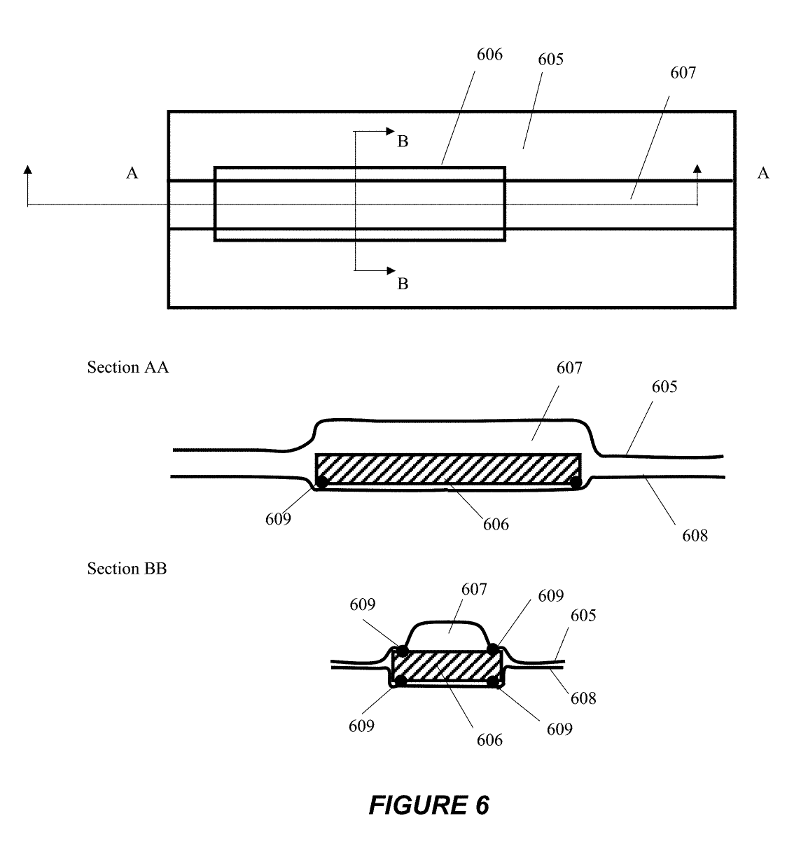

The present invention may also include a system and method including a compartment in a pouch, depressors and a magnet. The compartment from the previous paragraph may have a magnet disposed to one side of the compartment to collect magnetic particles inside the compartment against the inside wall of the compartment opposite to the magnet. The magnet may be moveable so that magnetic particles may be collected and then brought to desired portions of the compartment. While simultaneously deploying the magnet as well as the system of depressors, the particles of the compartment may be concentrated and/or the content of the compartment may be reduced in volume. The particles and/or the contents of the compartment may also be brought near to a desired edge of the compartment by using the depressors and moving the magnet. FIG. 5 shows an example of system and method that includes a compartment (512) in a pouch, a depressor (515) and a magnet (514) to concentrate magnetic particles (513).

This invention may also include a flow channel with a rigid side and a flexible side called “rigid-flexible channel” that comprises a piece of material having two sides and located between a first and a second film, the first side of the piece of material is bonded to the first film in such a manner that flow does not contact that side of the piece of material, wherein the second side of the piece of material is either not bonded or bonded to one or more regions of the second film in such a manner that a flow of liquid or gas between the two films will contact the second side of the piece of material forming a capillary. The rigid-flexible channel can also be made by sealing a single film to a piece of material in such a manner that a channel is formed between the film and the material.

In one preferred embodiment, the rigid-flexible channel is formed by first inserting a piece of material into a compartment. The piece of material has a cuboid shape and its thickness is preferably less than about 10 millimeters. One surface of this material placed inside of the compartment is sealed to one adjoining layer of plastic or film and the other side is not sealed, or it is sealed is such a way that a channel is formed.

When fluid pressure increases in the rigid-flexible channel, the fluid pressure causes the film of the channel to rise up above the surface in an amount that depends on the fluid pressure in the capillary to create a capillary flow channel over that side of the plastic piece.

The plastic piece can be first chemically or biologically functionalized so that desired reactions with materials contained in the fluid flowing over the surface of this plastic piece may be allowed to take place.

This invention may include a rigid-flexible channel where fluid pressure of a predetermined amount is used to cause the film over the plastic piece to rise by a known amount to achieve a desired height of the capillary channel. A differential pressure is then applied between the inlet end and the outlet end of the capillary while still maintaining the same average pressure in the capillary. The difference in pressure between the inlet end of the capillary and its outlet end causes a fluid flow through the capillary channel of a known amount and in a known direction. The average fluid pressure in the capillary and the differential pressure are established and controlled to achieve the desired capillary height as well as the desired flow rate through the capillary.

The thickness and type of plastic film of the flexible film that forms the rigid-flexible channel can be chosen to have suitable elastic properties, such as elastic modulus, so that the desired capillary height is achieved for a desired value of fluid pressure.

The channel dimensions of the rigid-flexible channel can be set to allow a suitable flat region with respect to capillary height to be created that will permit analysis and observations within the channel to proceed without undue complications that may otherwise arise.

This invention also includes a pouch formed from one or more pieces of film, wherein the pouch comprises:

a) a first region where two portions of film have been sealed together, the first region having a first and second side; and

b) a second region defining a channel where the two portions of film have not been sealed together, the second region having a first and second side; and

c) a third region that defines a portion of the channel, wherein two sides of the channel are formed from one or more pieces of a film that can have a greater rigidity than the portions of film that form the first and second regions.

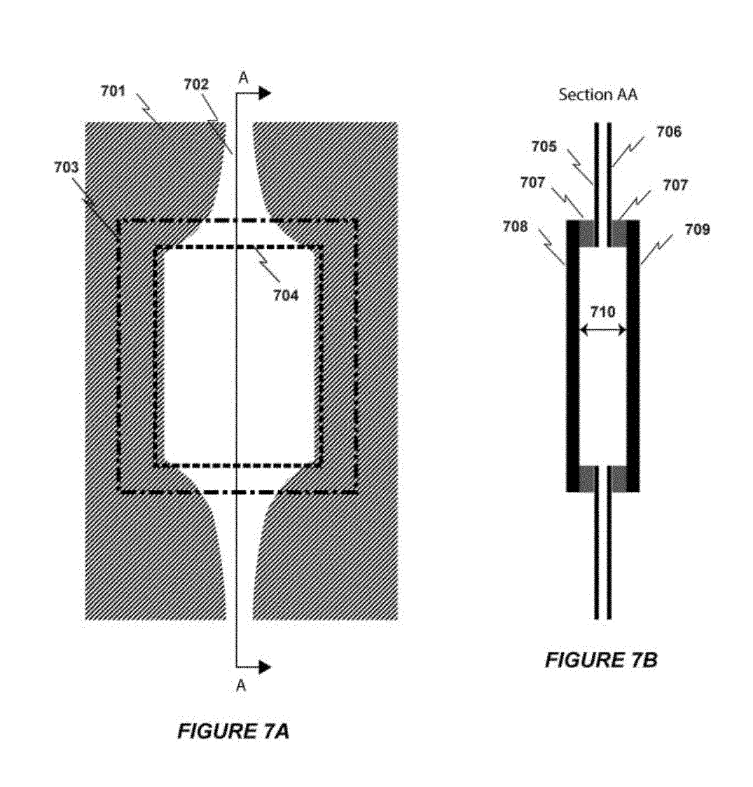

The channel of this pouch can be generated by replacing the films in a certain region of the channel with new films, wherein the new films may have a higher rigidity than the original sheets. This type of channel can be easily manufactured by cutting a window on the pouch over the channel and then gluing two new pieces of material at both sides of the window. One or both of new pieces of material may be rigid. These channels are ideal when a rigid surface is needed on a flow channel, for example, to attach molecules and conduct optical measurement. The pieces of material may be optically transparent and have better optical properties than the rest of the pouch. The new pieces of material may also have chemical properties that are different from the rest of the pouch and can be functionalized for attachment of specific molecules. FIG. 7 shows an example of such a channel.

The distance between the surfaces that form a channel in channels that have a rigid surface influence the channel section area and therefore the relation between total flow and local fluid velocity in the capillary. Furthermore, the distance between the surfaces in a capillary that lays horizontally determines the distance that particles sedimenting in the capillary need to travel before reaching the bottom surface. Preferably, the distance between the surfaces in flow channels that have at least one rigid surface is between 50 microns and 1,000 microns, or more preferably, between 100 and 400 microns.

Materials and films of the pouches of this invention can be made from optically transmissive materials to aid in optical detection within the regions of the capillary.

In exemplary embodiments, the rigid materials used in flow channels and compartments of the present invention may be composed of glasses, inorganic glasses, plastics, including acrylics, polystyrene and copolymers of styrene, polypropylene, polyethylene, polybutylene, polyurethanes, Teflon, polysaccharides, nylon or nitrocellulose, resins, and other polymers, carbon, metals, ceramics, silica or silica-based materials including silicon and modified silicon and silicon wafers. In some applications the material can be a composite material.

In exemplary embodiments, the film that forms the flow channel may be composed of plastics, including polyester, acrylics, polystyrene and copolymers of styrene, polypropylene, polyethylene, polybutylene, polyurethanes, Teflon®, Mylar®, polysaccharides, nylon or nitrocellulose, resins, and other polymers.

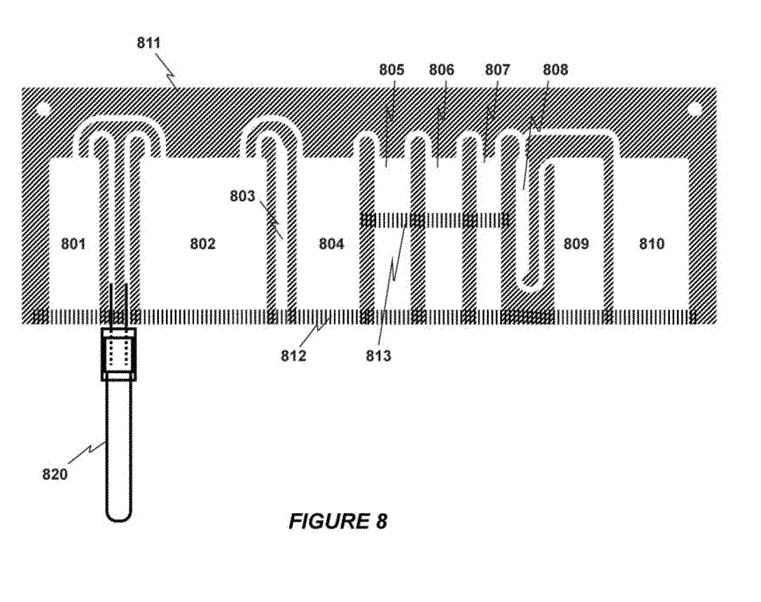

In one example, a pre-treated plastic bar is inserted into the capillary compartment (808) in FIG. 8 prior to sealing. This plastic bar is heat sealed to only one of the two films to permit fluid to flow over the unsealed surface of the plastic bar. Pressure from the incoming fluid flow will cause the unsealed plastic surface to rise to a height of about 0.05-1 millimeter by adjusting the flow pressure to a value to achieve this degree of rise. In an exemplary embodiment, the surface of the plastic bar is functionalized to have certain properties.

Surfaces of the materials used in this invention can be functionalized with molecules by physical or chemical adsorption. In preferred embodiments, the surfaces of the piece of material that forms the flow channel is functionalized with one or more biomolecules. Such methods of functionalization are known in the art. For instance, a gold surface can be functionalized with nucleic acids that have been modified with alkanethiols at their 3′-termini or 5′-termini. See, for example, Whitesides, Proceedings of the Robert A. Welch Foundation 39th Conference On Chemical Research Nanophase Chemistry, Houston, Tex., pages 109-121 (1995). See also Mucic et al., Chem. Commun. 555-557 (1996) (describing a method of attaching 3′ thiol DNA to flat gold surfaces; this method can be used to attach oligonucleotides to nanoparticles). The alkanethiol method can also be used to attach oligonucleotides to other metal and semiconductors. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes).

Oligonucleotides terminated with a 5′ thionucleoside or a 3′ thionucleoside may also be used for attaching oligonucleotides to solid surfaces. Another example of surface functionalization that may be used in the present invention is the immobilization of antibodies and other binding members to the surface either by physical adsorption or by direct or indirect chemical linkage. For instance, surfaces can be functionalized by chemically linking streptavidin molecules to them, which are capable of coupling to probes comprising one or more biotin molecules. The following reference describes the attachment of biotin labeled oligonucleotides to a streptavidin functionalized surface. Shaiu et al., Nucleic Acids Research, 21, 99 (1993). Digoxigenin and anti-Digoxigenin antibodies can also be used to attach probes to surfaces.

The surfaces can be functionalized by a monolayer of one or more molecules. Methods of producing self-assembled monolayers are well known in the art. In particular, there are several known methods to assemble monolayers of thiolates on metal surfaces. See e.g., Love, J. C. et al., Chem. Rev., 105, 1103 (2005).

The surface functionalization methods described above can be used to couple molecules that prevent or reduce non-specific interactions with the surface. For instance, after immobilization on to the surface of an analyte binding molecule, such as a ssDNA or an antibody, physical adsorption on the surface of a protein that blocks non-specific interactions is often conducted. Common proteins used as blockers are: bovine serum albumin (BSA), fish serum and milk proteins, such as casein.

The following references describe other methods that may be employed to attach oligonucleotides to surfaces: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langrnuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

In one example, air in compartment (801), FIG. 8, is forced to flow out of one needle and into the external container that contains a sample (820) by applying pressure to the compartment using one of the known methods. In one example, the sample is whole blood and is provided in a Vacutainer that is designed with a rubber seal that makes an air tight seal to the two needles that have penetrated it. This air displaces blood in the vacutainer and forces it to move from the Vacutainer to compartment (802).

In one embodiment, compartment (802) accepts a sample of between 3 and 15 milli-liters of blood from the vacutainer. Preferably, compartment (802) is pre-loaded with buffer that will aid in the lysing of cells.

In another embodiment, after cell lysing is complete, the contents of pouch compartment (803) are allowed to enter compartment (802). Compartment (803) may contain substances that aid in concentrating the sample. For example, compartment (803) may contain magnetic beads affixed with certain probes that are used in the next process step to wash and then concentrate the sample.

Various methods of isolating an objective biological substance from a sample containing a biological substance, such as nucleic acid, protein, carbohydrate and the like, have been conventionally studied. As examples of such methods, methods comprising extraction and separation using organic solvents, methods of isolating the objective substance using a molecule filter based on the size of the molecule, isolation methods utilizing a carrier capable of reversibly binding with a particular biological substance, and the like are known. Of the above-mentioned methods, isolation methods utilizing a carrier have many advantages for simultaneous processing of a number of samples. Particularly, isolation utilizing a magnetic carrier is highly convenient. This is because a biological substance—magnetic carrier complex can be collected by applying a magnetic field, and a collecting device, such as centrifuge and the like, is not required.

Methods of isolating a biological substance utilizing a magnetic carrier have been specifically developed as isolation methods of various nucleic acids.

An example of a magnetic carrier are particles with iron oxide having a coating of polymerizable silane capable of covalent binding with affinity molecules (e.g., nucleic acid and the like). These particles require a silane coating that binds with affinity molecules (e.g., nucleic acid). As an improvement of the above, spherical magnetic silica particles containing superparamagnetic metal oxide are known. Such magnetic silica particles are complexes of superparamagnetic metal oxide with inorganic porous wall substance consisting of fine silica particles and have a specific surface area of about 100-800 m²/g. Preferable superparamagnetic metal oxide content is about 10-60 wt %, and preferable particle size is about 0.5-15 microns.

In addition, magnetic silica particles having a structure, in which plural fine core particles comprising metal or metal oxide consisting of multiple magnetic domains coated with a film or fine particles of silicon oxide, have been also proposed.

Moreover, an analysis method and a device using biological substances bound with a substance having magnetic reactivity are also known (see WO86/05815). According to this method, a magnetized particle or a magnetizable particle coated with a substance capable of binding with a single strand nucleic acid is used to separate and detect a single strand nucleic acid. Specifically, nitrocellulose, which is one kind of cellulose derivative, is coated on the surface of a magnetized particle, and nitrocellulose and single strand nucleic acid of DNA or RNA are specifically bound with each other. According to this method, since a single strand nucleic acid of DNA or RNA is specifically bound with the magnetic carrier, the surface of the magnetized particle needs to be covered with a cellulose derivative such as nitrocellulose and the like.

Another example of magnetic carriers are monodisperse superparamagnetic beads produced according to EP 83901406.5, the disclosure of which is incorporated herein by reference. In these beads, the iron is very uniformly distributed and provides a very uniform response to a magnetic field which is important in designing a reproducible procedure, particularly for automation, since all the beads move at the same speed. Furthermore, since a reproducible amount of iron can be incorporated in each particle, this can be adjusted to a relatively low level which permits the specific gravity of the particles to be in the range of about 1 to 2. Advantageously, the monodisperse particles are spherical beads having a diameter of at least about 0.5 micron, being preferably not more than about 10 microns. Smaller particles sediment more slowly and in some cases the sedimentation time may be long compared to the reaction time, thus avoiding the need for physical agitation. The attachment of the probes to the particles may be by direct chemical bonding as well as affinity binding, by streptavidin/biotin complexes and the like. For attachment of the probes, the magnetic particles may carry functional groups such as hydroxyl, carboxyl, aldehyde or amino groups. These may in general be provided by treating uncoated monodisperse, superparamagnetic beads, to provide a surface coating of a polymer carrying one of such functional groups, e.g. polyurethane together with a polyglycol to provide hydroxyl groups, or a cellulose derivative to provide hydroxyl groups, a polymer or copolymer of acrylic acid or methacrylic acid to provide carboxyl groups or an aminoalkylated polymer to provide amino groups. U.S. Pat. No. 4,654,267 describes the introduction of many such surface coatings.

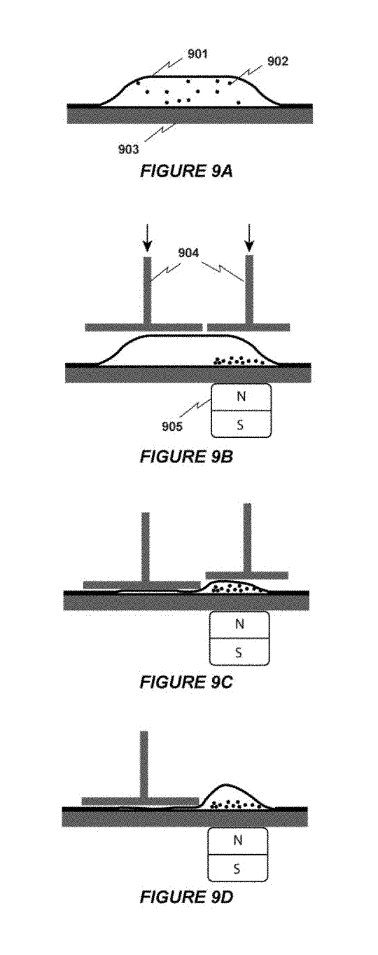

In another embodiment of this invention, a magnet is brought to the surface of compartment (802) to attract the magnetic beads to its surface. The contents of compartment (802) other than the magnetic particles that are held against the surface by the magnet are then pushed into compartment (801), which now serves as a waste compartment, since its use as an air pump is now over. Depressors may compress compartment (802) and actuators open the respective flow channels to permit fluid to flow to compartment (801) using methods that are known in the art, such as the depressors (904) shown in FIG. 9.

In another embodiment, referring to FIG. 8, wash buffer from compartment (804) may be brought into compartment (802) by removing its magnet and depressor while depressing compartment (804). The contents of compartment (802) are now allowed to be washed and then the magnet may be brought to the surface of compartment (802) to collect the beads for a second time. The contents of compartment (802) may then be pushed into compartment (801) to complete a second wash cycle, while holding the magnetic beads to the inner surface of compartment (802) and not allowing them to flow to compartment (801). A similar third wash cycle may be performed if desired.

Further referring to FIG. 8, in another embodiment the beads can be concentrated, reduced in volume and the target molecules bound to the beads brought into the small compartment (805). This can be done in a series of steps that will be described next. The remaining contents of compartment (804) are pushed into compartment (802) and the magnet on compartment (802) is removed. A split depressor acting on compartment (804) is allowed to have its top half released while keeping its bottom half depressing compartment (804). This effectively cuts the volume of compartment (804) in half. The contents of compartment (802) are now pushed into the upper half of compartment (804). The volume of fluid in compartment (802) that was pushed into it from compartment (804) is just enough to fit within the upper half of compartment (804) when it is pushed back in. Compartment (802) is then completely depressed to force all of its contents including the magnetic beads into the upper half of compartment (804). A magnet positioned close to the top of compartment (804) now collects the beads and the volume of fluid in compartment (804) is pushed back into compartment (802) while retaining the magnetic beads in compartment (804). Compartment (805) contains an elution buffer and its contents are pushed into compartment (804)'s upper half while removing the magnet from compartment (804) to allow mixing of the magnetic beads with the elution buffer. Heater coils disposed to the surface of compartment (804) are then activated to raise the temperature of compartment (804) to make the target elute from the magnetic beads.

The magnet is again deployed to capture the beads in compartment (804) and the contents of the upper half of compartment (804) are then pushed into compartment (805). This contains what is termed the supernatant that has the target material, but the magnetic beads remain in compartment (804). Compartment (805) now has concentrated target material ready for further processing steps.

The contents of compartment (805) may then be pushed into compartment (806) for binding to the next reagent. When that step is complete, the contents of compartment (806) are pushed into compartment (807) to react with the final set of reagents. When this is complete, the contents of compartment (807) are in a state to be analyzed in the flow channel of compartment H.

Before this can happen, the flow channel may be prepared. Buffer solution is made to flow from compartment (810) into flow channel (808) and then through to the flow channel buffer exit compartment (809).

Contents of compartment (807) are made to flow into flow channel (808) and optionally held for a period of time to allow its contents to react with the surface of the flow channel.

Buffer from compartment (810) may be made to flow to wash out any unreacted material from the flow channel (808) and deposit this into compartment (809). After target material reacts to the surface of flow channel (808) any excess unreacted material has been pushed out to compartment (809).

Next, the capillary buffer supply compartment (810) may be depressed in a controlled manner to achieve a desired flow rate through the flow channel (808). Target flow rate is typically in the range of about 100 to 4000 micro-liters per minute. While maintaining a constant flow rate, optically detect the image of the center portion of the flow channel (808) by, e.g., optical means.

If desired, the direction of flow through the flow channel (808) may be reversed to image the center portion of the flow channel while flow is in the opposite direction. These two steps may be repeated several times to obtain several sets of data for analysis. This completes the diagnostic protocol. The images are analyzed to determine if the target molecule was present in the sample and estimate its concentration.

Optionally, the clamp may be replaced to seal the pouch for disposal so that contamination will be avoided during disposal of the used pouch.

A pouch with two or more compartments as the ones described in this invention can be used to conduct a complete medical diagnostic assay in a closed manner that prevents contamination of either the sample to be analyzed, the reagents contained in the pouch or the outside world. The pouches of this invention can substantially prevent any material other than the sample to be analyzed to enter the pouch and can also prevent anything in the pouch after an analysis to be released to the environment.

In another aspect, this invention includes a process to analyze a sample solution. A sample previously loaded in a pouch is subjected to a process that lyse the cells that contain the target molecule making the target accessible. Cell lysing can be conducted with several methods known in the art, including exposure to detergents, bead beating, ultrasound and heating. See for example Enzyme and Microbial Technology, Volume 8, Issue 4, April 1986, Pages 194-204; Journal of Microbiological Methods, Volume 81, Issue 2, May 2010, Pages 127-134; Integr. Biol., 2009, 1, 574-586. Then, functionalized magnetic beads are incubated with the lysed sample and the functionalized magnetic beads bind to the target molecule if the target molecule is present. The magnetic beads are washed by applying a magnet that collects the beads against the wall of the compartment and then removing at least some of the solution, optionally adding a new buffer solution and optionally removing the magnet. If less volume of buffer is added than the volume removed of the original solution, the beads and therefore also the target are concentrated. If the compartment has a flexible wall, the wall can be deformed to reduce the volume of the compartment. Multiple washes and concentration steps may be conducted to reach the desired purity and concentration. Then, the target can be eluted from the beads into an elution buffer by exposing the beads to conditions that reduce the affinity of the target for the beads. For example, if the beads are bound to the target through nucleic acid hybridization, the temperature can be raised or the salt concentration reduced. After the targets have dissociated from the beads, the magnet is applied to collect the beads in the compartment wall and the elution buffer is removed. Once the target molecule is in the elution buffer, different methods known in the art can be used to detect the target molecule, including the polymerase chain reaction (PCR) and non-enzymatic methods, such as enzyme-linked immunosorbent assay, or ELISA. In a preferred embodiment, the present devices and methods may be used to practice the assays described in co-pending published application US 20160258003 A1, the contents of which is incorporated herein by reference.

What is claimed is:

1. A pouch for use in a process for detecting the presence or concentration of a target analyte in a sample, wherein the pouch comprises:
a) one or more compartments capable of holding a fluid; and
b) one or more flow channels having at least two surfaces, wherein one flow channel i) connects two compartments within the pouch to permit fluid to flow from one compartment to another, and/or ii) connects one compartment of the pouch with the exterior of the pouch to allow fluid to flow from the compartment to the exterior of the pouch;
wherein at least one of the flow channels has a shape such that the channel can be closed with a single device that exerts pressure on such channel.

2. The pouch of claim 1, wherein the pouch is made by heat-sealing one or two films.

3. The pouch of claim 1, wherein at least one of the flow channels has a substantially U-shape, and wherein the single device comprises a clamp that exerts pressure on at least two surfaces of the flow channel.

4. The pouch of claim 1, wherein the pouch comprises at least five compartments and at least five flow channels, and wherein the single device is associated with the pouch and is capable of closing the at least five flow channels at the same time.

5. A pouch for use in a process for detecting the presence or concentration of a target analyte in a sample, wherein the pouch comprises:
a) a first compartment in first fluid connection with a first fluidic connector, wherein the first compartment is capable of holding a fluid and wherein fluid is capable of flowing between the first fluidic connector and first compartment; and
b) a second compartment in second fluid connection with a second fluidic connector, wherein the second compartment is capable of holding a fluid and wherein fluid is capable of flowing between the second fluidic connector and second compartment;
wherein the first and second fluidic connectors are positioned so as to be capable of simultaneously communicating with a fluid test sample.

6. The pouch of claim 5 wherein the first and second fluidic connectors are releasable fluidic connectors.

7. The pouch of claim 5 wherein the first and second fluidic connectors are hollow needles.

8. The pouch of claim 5 wherein the first and second fluidic connectors are Luer taper connectors.

9. The pouch of claim 5, wherein the pouch is made by heat-sealing one or two films.

10. The pouch of claim 7, wherein the needles are substantially parallel to each other.

11. The pouch of claim 7, wherein the needles are positioned to be from about 1 to about 30 millimeters apart.

12. The pouch of claim 5, wherein the first and second fluid connections are configured such that they are capable of being closed with a single device that exerts pressure on such fluid connections.

13. The pouch of claim 12, wherein the first and second fluid connections comprise substantially U-shaped channels.

14. A pouch for use in a process for detecting the presence or concentration of a target analyte in a sample, wherein the pouch comprises:
a) a first compartment in first fluid connection with a container external to the pouch, wherein the first compartment is capable of holding a fluid and wherein fluid is capable of flowing between the container and the first compartment; and
b) a second compartment in second fluid connection with the container, wherein the second compartment is capable of holding a fluid and wherein fluid is capable of flowing between the container and the second compartment;
wherein the first and second fluid connections are positioned so as to be capable of simultaneously communicating with a fluid that may be present in the container.

15. The pouch of claim 14, wherein the container comprises a cap that creates an air tight seal with the container.

16. A pouch formed from one or more pieces of film, capable for use in a process for detecting the presence or concentration of a target analyte in a sample, wherein the pouch comprises:
a) a first region where two portions of film have been sealed together, the first region having a first and second side; and
b) a second region defining a channel where the two portions of film have not been sealed together, the second region having a first and second side; and
c) a third region that defines a portion of the channel, wherein two sides of the channel are formed from one or more pieces of a film having a greater rigidity than the portions of film that form the first and second regions.

17. The pouch of claim 16, wherein at least a portion of the film having a greater rigidity is substantially transparent.

18. The pouch of claim 16, wherein at least a portion of the film having a greater rigidity is functionalized with one or more biomolecules on the surface that faces the interior of the pouch.

19. The pouch of claim 16, wherein the film having a greater rigidity is affixed to the pouch using two-sided tape.

20. The pouch of claim 16, wherein the thickness of the portion of the channel defined by the third region is from about 50 to about 1,000 microns.

21. A method for loading a sample from a container into a pouch, the method comprising:
a) providing the pouch of claim 5;
b) connecting the first and second fluidic connectors from the pouch to fluidic connectors fluidically connected to the interior of an external container that contains a sample; and
c) applying pressure to the first compartment to initiate flow of material from the first compartment into the external container and to initiate flow of material from the external container into the second compartment.

22. A method for loading a sample from a container into a pouch, the method comprising:
a) providing the pouch of claim 7;
b) providing a container that contains a sample;
c) penetrating the container with the first and second needles; and
d) applying pressure to the first compartment to initiate flow of material from the first compartment into the external container and to initiate flow of material from the external container into the second compartment.

23. A method for loading a sample from a container into a pouch, the method comprising:
a) providing the pouch of claim 14 which has a sample in the external container; and
b) applying pressure to the first compartment to initiate flow of material from the first compartment into the external container and to initiate flow of material from the external container into the second compartment.

24. A method to reduce the volume and exchange the liquid in which a magnetic particle is suspended, the method comprising:
a) providing a compartment having a flow channel, wherein the compartment contains an initial volume of liquid with at least one magnetic particle;
b) placing a magnet in sufficient proximity to the compartment to attract the magnetic particle, optionally waiting until the particle has reached a region close to the magnet;
c) reducing the volume of the compartment to remove at least a part of the liquid from the compartment while keeping the magnetic particle within the compartment;
d) constraining at least part of the compartment such that the maximum volume that the compartment can reach is less than the initial volume of liquid;
e) adding fresh liquid to the constrained compartment; and
f) causing the liquid and the magnetic particle to leave the compartment.
